(19) United States
(12) Patent Application Publication
(10) Pub. No.: US 2024/0159704 A1
(43) Pub. Date: May 16, 2024

(54) COMPOSITE MEMBRANE FOR WESTERN BLOTTING CONTAINING A PVDF NANOFIBER WEB AND MANUFACTURING METHOD THEREOF

(71) Applicant: AMOLIFESCIENCE CO., LTD., SEOUL (KR)

(72) Inventors: Chan KIM, Gwangju-si (KR); Eu Gene CHO, Muan-gun (KR); Sang Chul SUH, Seoul (KR); In Yong SEO, Seoul (KR)

(21) Appl. No.: 18/423,282

(22) Filed: Jan. 25, 2024

Related U.S. Application Data
(62) Division of application No. 16/208,968, filed on Dec. 4, 2018, now abandoned.
Application No. 16/208,968 is a continuation of application No. 14/242,036, filed on Apr. 1, 2014, now abandoned.
Application No. 14/242,036 is a continuation-in-part of application No. PCT/KR2012/008013, filed on Oct. 4, 2012.

(30) Foreign Application Priority Data
Oct. 4, 2011 (KR) 10-2011-0100508

(51) Int. Cl. G01N 27/447; D01D 5/00; D04H 1/44; D04H 1/555; G01N 33/544

(52) U.S. Cl. CPC G01N 27/44739; D01D 5/003; D04H 1/44; D04H 1/555; G01N 33/544

(57) ABSTRACT

Provided is a composite membrane for western blot, in which the composite membrane is prepared by combining nanofiber webs with nonwoven fabrics, and a basis weight of the nanofibers is in a range of 1 gsm to 50 gsm on the nonwoven fabrics, and an average pore size is in a range of 0.1 μm to 1.0 μm. The composite membrane for western blot including nanofibers has advantages such as saving of a production cost, and an excellent response characteristic due to a capillary phenomenon of a double structure, to thereby easily detect even a small amount of a particular substance present in a protein.

### TECHNICAL FIELD

The present invention relates to a composite membrane for western blot containing polyvinilidenflouride (PVdF) nanofibers, and a manufacturing method thereof, and more particularly, to a composite membrane for western blot containing PVdF nanofibers, which is configured to have a composite of electrospun nanofiber webs and nonwoven fabrics, to thereby reduce a production cost, provide excellent response characteristics, and provide a good protein detection sensitivity, and a method of manufacturing the same.

### BACKGROUND ART

Western blot (or western blot analysis) is a technique of finding a particular protein from a mixture of several proteins. According to the western blot, proteins extracted from cells or tissues are mixed with sample buffers and the proteins mixed with sample buffers are put on a molecular sieve made of acrylamide, to then perform an electrophoresis and to thus make a material called SDS (sodium dodecylsulfate) or SDS-page contained in the sample buffers take negative electricity all over the proteins so as to make the proteins be attracted toward positive electricity. In this case, the molecular sieve prevents the proteins from proceeding to thus cause small size molecules to move quickly and large molecules to move slowly, and to thereby form bands in different sizes. Here, once a membrane is placed on gel of proteins separated depending on the size, and electricity is applied between the membrane and the gel, the proteins are transferred to the membrane in a separated state. Here, antibodies against specific proteins to be detected are coupled and unique secondary antibodies are re-coupled with the coupled antibodies against specific proteins. A reaction exhibited by the color development and fluorescence of the unique secondary antibodies re-coupled with the antibodies against specific proteins is processed by an X-ray imaging method, which is called the western blot.

In this case, raw materials of the membrane are nitrocellulose, nylon, polyvinilidenflouride (PVdF), etc., easy to perform hydrophobic interaction with protein. This membrane is manufactured in a manner such as a dry process, a wet process, a dry-wet casting process, by a phase separation method in which a solvent and a polymer are poured into a non-solvent such as water. However, since it is not easy to control the phase separation method due to the influence of complex factors involved in a phase conversion process, it was difficult to achieve membranes having a uniform pore size distribution. Further, since the shape of the pores is formed in the phase separation process, a two-dimensional closed structure (or closed pore) that is not connect from the surface to the back side is formed, to thereby make it difficult to expect a high porosity and a high specific surface area.

Recently, an electrospinning process which is one of membrane manufacturing methods, is a method of obtaining nanofibers of a three-dimensional non-woven fabric shape by using a polymer solution and a high voltage electric field. Such nanofibers have advantages that the structure of pores can be controlled by diameter of fibers and post-processing, and a high porosity and a high specific surface area can be provided.

Up to now, as a method of manufacturing a membrane for western blot by using nanofibers, Korean Patent Laid-open Publication No. 10-2011-0035454 entitled “Nanofiber membrane for western blot and its manufacturing method,” and Korean Patent Laid-open Publication No. 10-2011-0058957 entitled “Integral membrane for western blot and its manufacturing method” were proposed.

However, in the case of preparing membranes by using nanofibers alone, air bubbles are generated between the membranes and the gel from which proteins have been separated when performing western blotting. As a result, rigidity of the nanofiber membranes is so weak that it is not easy to remove the air bubbles. Also, when nanofiber membranes are put on gel, an attachment or overlapping phenomenon may appear mutually among nanofibers, by a flexibility degree of nanofibers and by electrostatic forces. In order to prevent this, it is necessary to maintain the thickness of the nanofiber membranes to be a certain thickness level or thicker, to thereby consequently cause material and process costs to rise.

In addition, in the case that the nanofibers are laminated with paper, since expansion rates of PVdF nanofibers and paper by the methanol differ from each other, during a methanol pretreatment process, a phenomenon of separating nanofibers from paper appears. In addition, since stiffness of paper is too large when compared to nanofibers, air bubbles are generated at a contact surface between the gel and the membranes. However, since it is not easy to remove the air bubbles, there is a problem that it is difficult to perform western blotting.

Thus, there has still been a need for a membrane for western blot having a uniform pore distribution, a high porosity, an easy removal of air bubbles, and a suitable flexibility.

The present invention has been proposed in this background, the present inventors have found that as a result of researches about the improvement of the aforementioned problems of the prior art, nanofiber webs and nonwoven fabrics are incorporated and made composite, to thereby find out this problem can be removed, and complete the present invention.

### SUMMARY OF THE INVENTION

To solve the above problems or defects, it is an object of the present invention to provide a composite membrane for western blot having advantages of more inexpensive features, easier handling features, and more excellent protein detection sensitivity features than the conventional art, by combining nanofiber webs with nonwoven fabrics into a composite.

To accomplish the above and other objects of the present invention, according to an aspect of the present invention, there is provided a method of manufacturing a composite membrane for western blot, the composite membrane manufacturing method comprising the steps of: dissolving a polyvinilidenflouride (PVdF)-based polymer material in a solvent to prepare a spinning solution; obtaining webs of PVdF-based polymer nanofibers from the spinning solution by an electrospinning method; and combining the resulting nanofiber webs with nonwoven fabrics to obtain a composite membrane for western blot.

Preferably but not necessarily, the PVdF-based polymer material comprises: a fluorinated polymer including PVdF consisting of a homopolymer and PVdF consisting of a copolymer, alone or in combination, but not particularly limited thereto.

Preferably but not necessarily, in the present invention, a basis weight of the nanofibers is in a range of 1 gsm to 50 gsm, and an average pore size is in a range of 0.1 μm to 1.0 μm.

Preferably but not necessarily, according to the present invention, the combining of the nanofiber webs with the nonwoven fabrics is achieved by laminating the nanofiber webs and the nonwoven fabrics, or directly spinning the nanofibers on the nonwoven fabrics.

Preferably but not necessarily, the combining of the nanofiber webs with the nonwoven fabrics is achieved with any one method selected from squeezing, pressing, calendering, rolling, thermal bonding, and ultrasonic bonding.

Preferably but not necessarily, the combining of the nanofiber webs with the nonwoven fabrics may be performed while accompanying a heat treatment at 60° C. to 200° C.

According to another aspect of the present invention, there is provided a composite membrane for western blot, the composite membrane that is prepared by combining nanofiber webs manufactured by an electrospinning method with nonwoven fabrics, wherein the content of the nanofibers is in a range of 1 gsm to 50 gsm, and an average pore size is in a range of 0.1 μm to 1.0 μm.

Preferably but not necessarily, the solvent for use in the invention is one or more selected from the group consisting of di-methylformamide (DMF), di-methylacetamide (DMAc), THF (tetrahydrofuran), acetone, alcohol, chloroform, DMSO (dimethyl sulfoxide), dichloromethane, acetic acid, formic acid, NMP (N-Methylpyrrolidone), and fluorinated alcohols.

Preferably but not necessarily, the spinning method is one or more selected from the group consisting of electrospinning, electrospray, electrobrown spinning, centrifugal electro spinning, and flash-electro spinning.

Preferably but not necessarily, the nonwoven fabrics are one or more selected from the group consisting of PET (Polyethylene terephthalate), PP (polyprophylene), PE (polyester), nylon, cellulose-group, and PVdF-group, which are not particularly limited to the thickness or diameter of the fibers.

Preferably but not necessarily, the fiber diameter of the nonwoven fabrics is in a range of 10 μm to 100 μm, particularly preferably, 60 μm to 70 μm, which can be prepared in various methods including melt-blown, spun-bond, flash spinning, and sea-island (or sea island cotton yarn type).

As described above, according to the present invention, it is possible to provide a composite membrane for western blot, to reduce a production cost, as well as to provide an excellent protein detection sensitivity and an improved handling convenience, in comparison with the case of using nanofiber webs alone by a capillary action according to lamination of the nanofiber webs and nonwoven fabrics.

### DESCRIPTION OF DRAWINGS

FIG. 1 is a photographical view showing scanning electron micrographs of a PVdF nanofiber web that is prepared according to an embodiment of the present invention, in which the micrograph (a) shows the scanning electron micrograph of the PVdF nanofiber web when a basis weight of the PVdF nanofiber web is 7 grams per square meter (gsm), the micrograph (b) shows the scanning electron micrograph of the PVdF nanofiber web when a basis weight of the PVdF nanofiber web is 9 gsm, and the micrograph (c) shows the scanning electron micrograph of the PVdF nanofiber web when a basis weight of the PVdF nanofiber web is 14 gsm;

FIG. 2 is a photographical view showing scanning electron micrographs of cross sections of a PVdF nanofiber web that is prepared according to an embodiment of the present invention and that is laminated with a PET nonwoven fabric in which the micrograph (a) shows the scanning electron micrograph of the laminated result when a basis weight of the laminated result is 7 gsm, the micrograph (b) shows the scanning electron micrograph of the laminated result when a basis weight of the laminated result is 9 gsm, and the micrograph (c) shows the scanning electron micrograph of the laminated result when a basis weight of the laminated result is 14 gsm;

FIG. 3 is a photographical view showing scanning electron micrographs in which the micrograph (a) shows the scanning electron micrograph of PET nonwoven fabrics that are used in the present invention, and the micrograph (b) shows the scanning electron micrograph of PVdF nanofiber webs that are prepared by the present invention and that are laminated with PET nonwoven fabrics;

FIG. 4 is a graph showing the results of PMI (Positive Material Identification) tests of a composite membrane prepared according to an embodiment of this invention;

FIG. 5 is a photographical view showing results of western blot by using a composite membrane prepared according to an embodiment of the present invention; and

FIG. 6 is a photographical view showing results of western blot by using a composite membrane prepared according to an embodiment of the present invention and a membrane according to a comparative example of the present invention.

### DETAILED DESCRIPTION OF THE INVENTION

The objects, features and advantages of the invention will become apparent through the exemplary embodiments that are illustrated in the accompanying drawings and detailed in the following description. Accordingly, the inventive technological concept can be made by those skilled in the art without departing from the spirit and scope of the invention.

A composite membrane for western blot according to an embodiment of the present invention is prepared by a method including the steps of: dissolving a PVdF-based polymer first in a suitable solvent to prepare a spinning solution in a concentration capable of being subject to a spinning operation; transferring the spinning solution to a spinneret; applying a high voltage to nozzles of the spinneret; spinning the spinning solution into nanofiber webs by using an electrospinning method; and laminating the electrospun nanofiber webs with nonwoven fabrics, or a method of directly electrospinning PVdF-based nanofibers on nonwoven fabrics, in which a basis weight of the nanofibers is in a range of 1 gsm to 50 gsm, and an average pore size is in a range of 0.1 μm to 1.0 μm.

Each step will be described below in detail.

### Preparing a PVdF-Based Nanofibers Spinning Solution

In the present invention, a fluorinated polymer including for example PVdF consisting of a homopolymer and PVdF consisting of a copolymer, alone or in combination may be used as the PVdF-based polymer material. The spinning solution of a spinnable concentration is prepared by using a commonly usable solution as the suitable solvent.

In the preparation of the spinning solution, the content of the PVdF-based polymer material is suitably in a range of 5% to 50% by weight. However, in the case of less than 5% by weight, the PVdF-based polymer material is not formed into nanofiber webs but is sprayed in the form of beads. In this case, it is difficult to configure a membrane. In the meantime, in the case of more than 50% by weight, it may be difficult to form fibers due to high viscosity and poor spinnability. Thus, the spinning solution is made into a concentration with which it easy to form a fibrous structure, and then it is desirable to control morphology of the fibers.

### Forming Polymeric Nanofiber Webs

The prepared spinning solution is transferred a spin pack by using a metering pump. Here, a voltage is applied to the spin pack by using a high voltage control apparatus in order to carry out an electrospinning operation. In this case, it is possible to control the used voltage between 0.5 kV to 100 kV. A current collector plate may be connected to the ground or may be charged into a negative (−) pole, before being used, and it is preferable that the current collector plate be made of electrically conductive metal, release paper, nonwoven fabrics, and the like. In the case of the current collector plate, in order to smooth focusing of fibers during spinning, it is preferable that a suction collector be attached to the current collector plate.

In addition, it is preferable that a distance from the spinning pack to the current collector plate be controlled in a range of 5 cm to 50 cm. During spinning, it is preferable that a discharge amount per holcminute should be controlled in a range of 0.01 cc/holcmin to 5 cc/holcmin by using a metering pump, and spinning be carried out in an environment of relative humidity of 30% to 80% in a chamber that can regulate the temperature and humidity during spinning.

Combining Polymer Nanofiber Webs with Nonwoven Fabrics into a Composite

The thus-prepared PVdF nanofiber webs are integrated with nonwoven fabrics such as PET, PP, PE, nylon, cellulose-based, and PVdF-based nonwoven fabrics, and are laminated in various methods such as compression, rolling, thermal bonding, ultrasonic bonding, and calendaring, to thus prepare a composite membrane.

In this case, a basis weight of the nanofibers may be produced variously in a range of 1 gsm to 50 gsm. In the present invention, the term “basis weight” is a unit representing the content of nanofibers and expressed as gsm (gram per square meter). In the case of nanofibers of less than 1 gsm, the amount of PVdF nanofibers is too low, and thus there may be drawbacks that protein detection cannot be performed with high sensitivity. On the other hand, in the case of nanofibers of more than 50 gsm, there may be problems that a process cost may increase due to an expensive material cost rise.

Further, an average pore size of the nanofibers is suitable in a range of 0.1 μm to 1.0 μm. In case of the average pore size of less than 0.1 μm, the post-treatment costs may rise and the transfer time may delay. In case of the average pore size of more than 1.0 μm, since concentration of protein to be transferred is low, the detection sensitivity may fall, and thus there may be disadvantages that accurate analysis cannot be made.

At the time of combining the nanofiber webs with the nonwoven fabrics according to the present invention, it is preferable that thickness of layers of the nanofiber webs be in a range of 5 μm to 20 μm, preferably 10 μm to 15 μm. In the case of the thickness of less than 5 μm, since the nanofiber web layer is too thin, a phenomenon that bands may move towards the nonwoven fabrics occurs in an electrophoretic process, to thereby cause occurrence of the disadvantage that the detection sensitivity may drop. On the contrary, in the case of the thickness of more than 20 μm, there may no big problem, but there may be a burden of a cost increase.

Further, thickness of the nonwoven fabrics combined with the nanofiber webs may be in a range of 50 μm to 200 μm, preferably 100 μm to 200 μm. In the case that thickness of the nonwoven fabrics is less than 50 μm, since thickness of the nonwoven fabrics is too small, it tends to have poor handling characteristics. On the contrary, in the case of the thickness of more than 200 μm, the total thickness of the composite membrane becomes too large. This does not cause a major problem for western blot, but may cause an undesirable burden of a cost increase.

More preferably, the thickness ratio of the nanofiber webs to the nonwoven fabrics combined with the nanofiber webs is in a range of approximately 1/15 to 1/10.

On the other hand, since the commonly prepared nonwoven fabrics are processed by a sizing procedure, bubbles may occur at the time of an electrophoresis when the nonwoven fabrics are combined with the nanofiber webs without any pretreatment. Thus, it is necessary to pretreat the nonwoven fabric to be combined with the nanofiber webs. Pretreatment of the nonwoven fabrics is usually performed with acetone or IPA (isopropoylachol), and washed using distilled water.

Since the nanofiber webs are laminated with the nonwoven fabrics according to the present invention, a membrane having an excellent detection sensitivity by a capillary action in comparison with the case of the nanofiber webs alone, can be obtained.

In the meanwhile, in the present invention, a heat treatment process may involve according to necessity, when nanofiber webs are combined with nonwoven fabrics. It is preferable that the heat treatment should be performed in a temperature range of 60° C. to 200° C. at which polymer does not melt. In the case of less than 60° C., since the heat treatment temperature is too low, the fusing between the nanofibers is unstable, and thus separation proceeds between the nanofibers at the time of pretreatment of methanol before the western blot is carried out. As a result, it is difficult to perform proper western blot. In addition, when the heat treatment temperature exceeds 200° C., PVdF-based polymer constituting the nanofibers is partially melted and the pore structure is blocked. Accordingly, a transfer of proteins is not adequately achieved from a SDS-page and thus an accurate analysis is made difficult in some cases.

Hereinafter, embodiments of the present invention will now be described in further detail. These embodiments are only typical examples for illustrating the present invention, and it will be apparent to a person having an ordinary skill in the art that the scope of the present invention is not to be construed as being limited by these examples.

### Example 1

PVdF (Kynar 761) of 20% by weight consisting of a homopolymer that is a hydrophobic polymer, was dissolved in a solvent DMAc, to thus prepare a spinning solution. The prepared spinning solution is transferred to a spinning nozzle by using a metering pump, and an electrospinning is carried out at the room temperature and pressure, using an applied voltage of 25 kV, a distance of 20 cm between a spinneret and a current collector, and a discharge amount per hole·minute of 0.01 cc/hole·min. Basis weights of the electrospun PVdF nanofiber webs were made to be 7 gsm, 9 gsm, and 14 gsm, respectively.

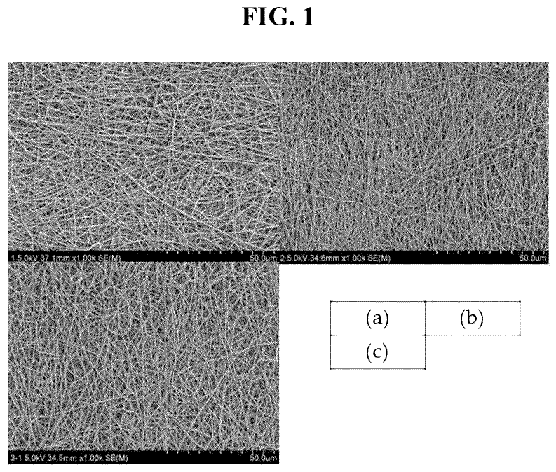

FIG. 1 illustrates scanning electron micrographs of electrospun PVdF nanofiber webs according to an embodiment of the present invention, respectively. As illustrated in FIG. 1, it can be verified that most fibers constituting the PVdF nanofiber webs show a diameter distribution in the range of 300 nm to 400 nm, and pores between the nanofibers and the nonwoven fabrics have a three-dimensional open pore (3-D open pore) structure to thus be uniformly opened from the surface to the back.

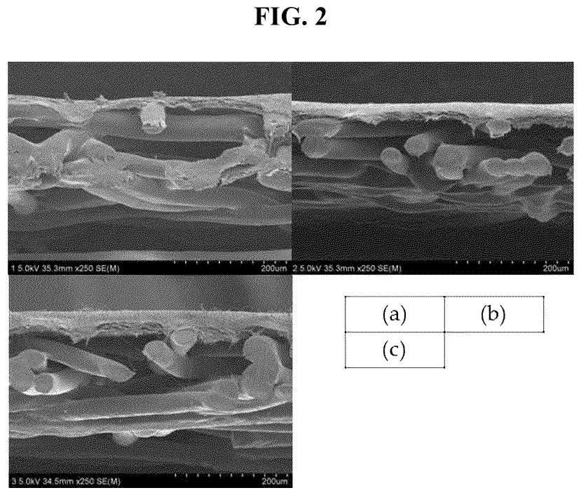

The thus-produced PVdF nanofiber webs were calendered and combined with PET nonwoven fabrics at 140° C., and a cross-sectional shape of a composite that is obtained by combining the PVdF nanofiber webs and the PET nonwoven fabrics was analyzed by a scanning electron microscope, which are shown in FIG. 2. As shown in FIG. 2, it can be seen that the PVdF nanofiber webs were combined with the PET nonwoven fabrics into a composite.

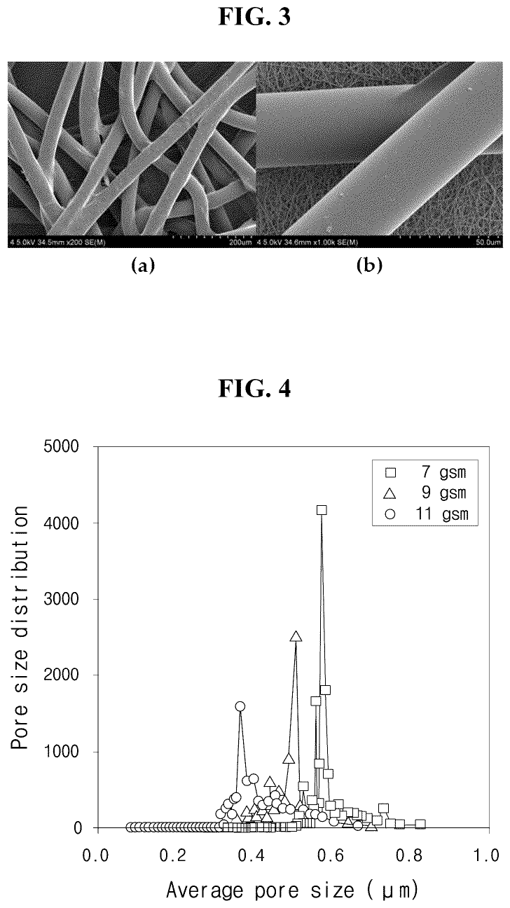

FIG. 3 is a photographical view showing scanning electron micrographs in which the micrograph (a) shows the scanning electron micrograph of the electrospun PVdF nanofiber webs, and the micrograph (b) shows the scanning electron micrograph of PET nonwoven fabrics, which are used in the present invention, respectively. From FIG. 3, it can be seen that the diameter of the PET nonwoven fabric is 20 μm approximately, and is 500 times as large as the diameter of the electrospun PVdF nanofiber web.

FIG. 4 is a graph showing results of analysis of a distribution of pores of a composite membrane laminated according to the present invention, by using PMI (Positive Material Identification) equipment such as a capillary flow porometer. As shown in FIG. 4, it can be seen that as the basis weight of the nanofibers increases from 7 gsm to 14 gsm, an average pore size is reduced. This is because the weight of the nanofibers increases.

### Example 2

Excepting that electrospun PVdF nanofiber webs are formed on PET nonwoven fabrics by carrying out electrospinning of PVdF nanofiber webs directly onto the PET nonwoven fabrics, a composite was obtained by combining the PVdF nanofiber webs with the nonwoven fabrics, in the same manner as that of Example 1, and then this composite was made to be subjected to calendering through a roller heated up to 140° C., to therefore prepare a composite membrane for western blot. It could be confirmed that the thus-prepared nanofiber had an average diameter of 400 nm to 500 nm similarly to that of Example 1, and the nanofiber was not shortened or desorbed but uniformly combined into a composite.

### Comparative Example

For comparison, western blotting was performed in the same manner as in Example 1 by using membranes consisting only of PVdF nanofiber webs having a basis weight of 70 gsm.

### Western Blot Test

A western blot test was carried out using samples of membranes prepared by Examples 1 and 2.

First, the sample prepared in Example 1 was pre-cut into pieces each of which has 8 cm×9 cm (length×width) in size, and immersed in a solution of 100% methanol for about 1 minute and activated so that the membrane can undergo hydrophobic interaction with respect to proteins in a gel.

The thus-activated membrane was transferred to a transfer buffer solution and was left alone for 10 minutes. Here, the transfer buffer solution was set to consist of 3.03 g/L trisma-base, 14.4 g/L glycine, and 20% methanol (200 ml/L). The gel to be transferred was fresh lightly dampened with the transfer buffer solution, and then placed on the membrane with care to avoid air bubbles. After the gel and the membrane were made to be in close contact, 3M® paper pre-wetted with the transfer buffer solution was put on both sides of the closely contacting gel and membrane to then be mounted in a transfer kit.

A transfer was conducted for 1 hour at 100 V using a mini-gel transfer kit, in which the transfer was carried out after a transfer tank had been put in ice to cut off heat generated during the transfer. After the transfer, the device was dismantled, and the membrane was separated and slightly pounded in TBST (tris-buffered saline with 0.05% tween 20). Here, the TBST consists of 0.2 M Tris pH 8 (24.2 g trisma base), 1.37 M NaCl (80 g NaCl), and adjust pH 7.6 to the desired value with concentrated HCl.

The total protein concentrations derived from oral epithelial cell carcinoma KB cell lines were 20 μg, 10 μg, 5 μg, 2.5 μg, 1 μg, and 10% SDS-page gel was used. Total transfer time was about 1 hour and 40 minutes, and the blocking time was 1 hour and 30 minutes.

The target protein to be detected was β-actin, and the first antibody was a β-actin antibody obtained from a mouse (santa cruz, sc-47778). These were diluted in a 1:5000 ratio, and were reacted with the transfer membrane at 4° C. for about a day, to thus obtain a first reaction result. Then, the first reaction result was reacted with goat anti-mouse IgG-HRP (santa cruz, sc-2005, which is an antibody made by injecting mouse immune globin into chlorine) that is a secondary antibody to which horseradish peroxidase (hydrogen peroxide-decomposing enzyme derived from horseradish) is bound, and then was put into reaction for one minute after a peroxide solution and a luminol enhancer solution (which emits fluorescence when luminol is oxidized by oxygen free radicals decomposed by the hydrogen peroxide-decomposing enzyme; LF-QC1010, ABFRONTIER, Korea) that are substrates for the horseradish peroxidase. β-actin protein expression was confirmed after exposing the transfer membrane having reacted with the substrate to an X-ray film for 2 minutes.

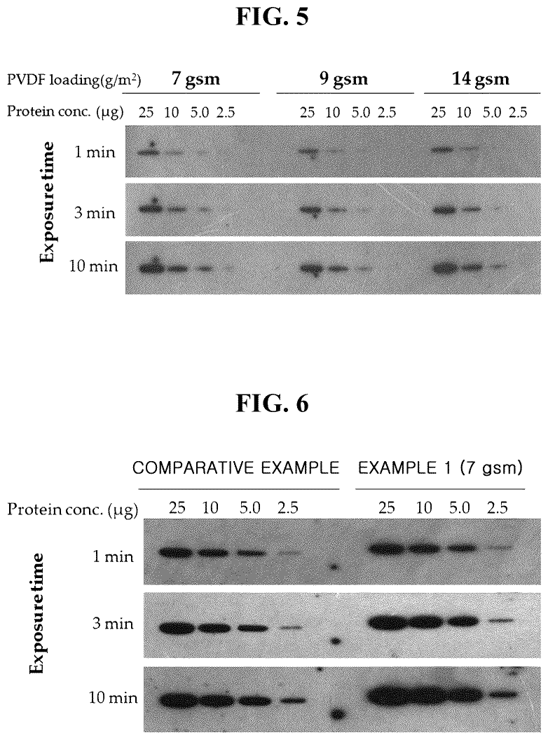

FIG. 5 is a photographical view showing results of western blot by using a composite membrane prepared according to Example 1 of the present invention. As shown in FIG. 5, although a basis weight of the nanofiber was changed into 7 gsm, 9 gsm, and 14 gsm in Example 1 of the present invention, respectively, the western blot results showed no significant changes, and showed the bands appeared in a clear and conspicuous manner in all the samples. However, in the case that the basis weight of the nanofiber was 7 gsm, detection bands appeared even at a pace where the protein concentration was relatively low but was about 2.5 μg. Accordingly, it could be confirmed that the detection sensitivity was excellent to some extent.

From these results, it can be seen that a problem of deterioration of a handling ability during western blotting is not caused even when there is a small basis weight of the nanofiber by combining nanofiber webs with nonwoven fabrics into a composite membrane according to the present invention, but in the case that the basis weight of the nanofiber is rather smaller, a porosity of the membrane is increased and thus the detection sensitivity of the protein is improved. Accordingly, according to the present invention, it is possible to provide a composite membrane for western blot, with a reduced process cost and at the same time an excellent detection sensitivity.

FIG. 6 is a photographical view showing results of western blot by using the nanofiber of 7 gsm of Example 1 according to the present invention, and the sample of a comparative example. As shown in FIG. 6, in comparison with the case of the membrane formed by the nanofibers alone (comparative example), a blotting size appeared relatively larger and more clearly in the present invention. From these results, compared with the comparative example, only a small amount of protein can be detected in the present invention, and thus it can be seen that the detection sensitivity for proteins in the present invention is more excellent than that of the comparative example.

As described above, the present invention has been described with respect to particularly preferred embodiments. However, the present invention is not limited to the above embodiments, and it is possible for one who has an ordinary skill in the art to make various modifications and variations, without departing off the spirit of the present invention. Thus, the protective scope of the present invention is not defined within the detailed description thereof but is defined by the claims to be described later and the technical spirit of the present invention.

The present invention may be applied to a composite membrane for western blot in which even a case that a small amount of a particular substance of a protein exists can be easily detected.

What is claimed is:

1. A composite membrane for western blot, the composite membrane comprising: a combination of a nanofiber web and a nonwoven fabric,
wherein a thickness ratio of the nanofiber web to the nonwoven fabric is in a range of 1/15 to 1/10, a content of the nanofiber web is in a range of 1 gsm to 50 gsm, and an average pore size of the nanofiber websis in a range of 0.1 μm to 1.0 μm.

2. The composite membrane of claim 1, wherein the nanofiber web comprise: a polyvinilidenflouride (PVdF)-based polymer material.

3. The composite membrane of claim 2, wherein the PVdF-based polymer material comprises: at least one selected from the group consisting of homopolymer PVdF and copolymer PVdF.

4. The composite membrane of claim 1, wherein fibers of the nonwoven fabric have a diameter in a range of 60 μm to 70 μm.

5. The composite membrane of claim 1, wherein the nonwoven fabric is formed of at least one selected from the group consisting of PET (Polyethylene terephthalate), PP (polyprophylene), PE (polyester), and nylon.

6. The composite membrane of claim 1, wherein a thickness of the nanofiber web is in a range of 10 μm to 15 μm, and a thickness of the composite membrane is in a range of 100 μm to 200 μm.

7. A method of detecting protein in a sample, the method comprising:
manufacturing a composite membrane:
wherein the manufacturing comprises:
dissolving a polyvinilidenflouride (PVdF)-based polymer material in a solvent to prepare a spinning solution;
electrospinning the spinning solution to obtain a nanofiber web, wherein the nanofiber web has a basis weight in a range of 1 gsm to 50 gsm, and an average pore size in a range of 0.1 μm to 1.0 μm; and
combining the nanofiber web with a nonwoven fabric to obtain the composite membrane, wherein fibers of the nonwoven fabric have a diameter in a range of 60 μm to 70 μm; and
performing a western blotting using the composite membrane, wherein the composite membrane is used in detecting protein in a sample.

8. The method of claim 7, wherein the PVdF-based polymer material comprises: at least one selected from the group consisting of homopolymer PVdF and copolymer PVdF.

9. The method of claim 7, wherein the nonwoven fabric is formed of at least one selected from the group consisting of PET (Polyethylene terephthalate), PP (polyprophylene), PE (polyester), and nylon.

10. The method of claim 7, wherein the combining comprises: laminating the nanofiber web on the nonwoven fabric.

11. The method of claim 7, wherein the electrospinning and the combining comprise: directly spinning the spinning solution on the nonwoven fabric.

12. The method of claim 7, wherein the combining is achieved with any one method selected from the group consisting of squeezing, pressing, calendering, rolling, thermal bonding, and ultrasonic bonding.

13. The method of claim 7, wherein the nanofiber web has a thickness in a range of 5 μm to 20 μm, and the composite membrane has a thickness in a range of 50 μm to 200 μm.

14. The method of claim 13, wherein the thickness of the nanofiber web is in a range of 10 μm to 15 μm, and the thickness of the composite membrane is in a range of 100 μm to 200 μm.

15. The method of claim 13, wherein a thickness ratio of the nanofiber web to the nonwoven fabric is in a range of 1/15 to 1/10.

16. The method of claim 7, wherein a thickness ratio of the nanofiber web to the nonwoven fabric is in a range of 1/15 to 1/10.
